(19) United States
(12) Patent Application Publication
(10) Pub. No.: US 2025/0275758 A1
(43) Pub. Date: Sep. 4, 2025

(54) System and Method for Adaptive-Color Imaging in Ultrasound Technology

(71) Applicant: FUJIFILM SonoSite, Inc., Bothell, WA (US)

(72) Inventors: Yong Zhou, Woodinville, WA (US); Bo Zhuang, Redmond, WA (US); Jean Tsou, Seattle, WA (US); Thomas Endres, Sagle, ID (US)

(73) Assignee: FUJIFILM SonoSite, Inc., Bothell, WA (US)

(21) Appl. No.: 18/592,070

(22) Filed: Feb. 29, 2024

(51) Int. Cl. A61B 8/08; A61B 8/00

(52) U.S. Cl. CPC A61B 8/5292; A61B 8/463; A61B 8/467

(57) ABSTRACT

Systems and methods for adaptive-color imaging in ultrasound technologies are disclosed. In aspects, adaptive-color imaging techniques are used for providing sensitive color images of internal body structures. The ultrasound system transmits waves reflecting off internal organs, with received signals processed into radiofrequency (RF) data. This RF data undergoes a color-imaging process to extract color-flow information, resulting in color-image data for ultrasound imaging. Feedback loops, including an RF-data loop, a color-image-data loop, or a combined loop, allow for continuous adjustments to parameters and filters in the imaging processes, thus refining images with each iteration. The adjustments for adapting parameters can be initiated on a user interface within the ultrasound system. Processors in the ultrasound system can also determine weighted data values for regions in an ultrasound image to evaluate cost functions used to adapt the parameters.

### BACKGROUND

Conventional ultrasound techniques include color-flow imaging, which is generally used for evaluating blood flow patterns within a body or system. This technique is typically used for vascular assessments and can visualize and quantify the direction and velocity of blood flow in a set area. Unlike the traditional black-and-white ultrasound images, color-flow imaging has a spectrum of colors to represent the movement of blood within vessels. In general, red and blue colors indicate blood flow in opposite directions, where red signifies flow towards the probe or transducer, while blue represents flow away from the transducer. Additionally, the shades of blue and red represent the velocity of blood flow where a lighter shade shows higher velocity, and a darker shade indicates a lower velocity.

However, in some cases, the effectiveness and precision of color-flow imaging is affected by limitations in the color sensitivity of ultrasound systems. The limitations in color sensitivity impact the ability to distinguish between slow and fast-moving blood, leading to potential inaccuracies. Consider, for example, a premature infant with very small and delicate vessels. Accurate color-flow imaging becomes difficult when trying to capture the subtle flow rates in such a small area. The limitations in color sensitivity impact the ability to assess blood flow with precision, especially in cases where the flow rates are relatively low or where intricate vascular networks need to be analyzed. In another example, consider a patient with poor blood flow in the veins. The low blood flow may result in poor color signals, which in turn can make it difficult to visualize the blood circulation. The existing color-flow imaging settings, including parameters like wall filter, gain, transmitting frequencies, and pulse repetition rate, offer some degree of customization. However, these settings prove insufficient when dealing with diverse patient populations. The variations in patients' anatomies and physiological conditions pose a significant challenge, as the standard color-flow imaging settings may not be universally applicable.

Additionally, the constraints in color sensitivity have impacts on the reliability of diagnostic interpretations. In clinical practice, the inability to accurately differentiate between various flow velocities can lead to misdiagnoses or incomplete assessments of vascular disorders. The limitations in color sensitivity interfere with the ultrasound system providing a complete and detailed analysis of blood flow patterns, thus resulting in poor user experiences for both the clinician and the patient.

### SUMMARY

Systems and methods for adaptive-color imaging in ultrasound technologies are disclosed. In aspects, adaptive-color imaging techniques are used for providing sensitive color images of internal body structures. The ultrasound system transmits waves that reflect off internal organs and processes received signals into radio frequency (RF) data. This RF data undergoes a color-imaging process to extract color-flow information, resulting in color-image data for ultrasound imaging. Feedback loops, including an RF-data loop, a color-image-data loop, or a combined loop, enable continuous adjustments to parameters and filters in the imaging processes, thus refining images with each iteration. The adjustments for adapting parameters can be initiated via a user interface within the ultrasound system. Processors in the ultrasound system can also determine weighted data values for regions in an ultrasound image to evaluate cost functions used to adapt the parameters.

In some aspects, a method is disclosed. The method includes receiving a first user selection that selects one or more processes from a process group. In addition, the method includes receiving a second user selection that selects feedback data from a data group. The method also includes adapting parameters of the selected one or more processes based on a cost function of the selected feedback data. Further, the method includes generating an ultrasound image having color-flow data based on the adapted parameters. Further, in response to generating the ultrasound image, the method includes generating similarity scores. In aspects, the similarity scores are based on comparing the ultrasound image to a plurality of additional ultrasound images. The method also includes selecting a target ultrasound image from the plurality of additional ultrasound images based on the similarity scores. In addition, the method includes obtaining parameter values of at least one of the one or more processes from the process group. Further, the method includes initializing an adaptation process with the obtained parameter values. The method also includes adapting the obtained parameter values through the adaptation process by using parameter values from the target ultrasound image. Additionally, the method includes generating an updated ultrasound image with color-flow data. In aspects, the generation of the updated ultrasound image is based on the adapted parameter values.

In some aspects, an ultrasound system is disclosed. The ultrasound system includes an ultrasound scanner configured to generate ultrasound images based on echoes of ultrasound signals transmitted by the ultrasound scanner into a subject at one or more anatomical targets of interest. In addition, the ultrasound system includes a display device configured to display a user interface. The ultrasound system also includes one or more processors and one or more computer-readable storage media having instructions stored thereon that, responsive to execution by the one or more processors, cause the one or more processors to determine weights for regions of a color box in an ultrasound image. The one or more processors are also configured to determine a cost function associated with the ultrasound image. In addition, the one or more processors are configured to evaluate the cost function associated with the ultrasound image. The one or more processors are also configured to determine values for parameters based on the evaluation of the cost function. Also, the one or more processors are configured to generate an additional ultrasound image with color-flow data. The generation of the additional image is based on the values of the parameters.

### BRIEF DESCRIPTION OF THE DRAWINGS

The appended drawings illustrate examples and are, therefore, exemplary embodiments and not considered to be limiting in scope. Throughout the drawings, the same numbers are used to reference like features and components.

FIG. 1 illustrates an example environment for an ultrasound system having an ultrasound scanner, in accordance with one or more implementations.

FIG. 2 illustrates an example implementation of the system from FIG. 1.

FIG. 3 illustrates an example of ultrasound-data processing in accordance with implementations disclosed herein.

FIG. 4 illustrates an example ultrasound RF-data image.

FIG. 5 illustrates an example ultrasound color-image-data image.

FIG. 6 illustrates an example initialization system for an adaptation process.

FIG. 7 illustrates an example implementation of a display of a user interface associated with an ultrasound system in accordance with one or more implementations.

FIG. 8 illustrates another example implementation of a display of a user interface associated with an ultrasound system in accordance with one or more implementations.

FIG. 9 illustrations an example implementation of a before-adaptation image and an after-adaptation image associated with an ultrasound system.

FIG. 10 depicts a method for generating ultrasound images based on adapted parameters.

FIG. 11 depicts another method for generating ultrasound images based on adapted parameters.

FIG. 12 depicts a method for generating ultrasound images based on a cost function of selected feedback data.

FIG. 13 illustrates a block diagram of an example computing device that can perform one or more of the operations described herein, in accordance with some implementations.

### DETAILED DESCRIPTION

Limitations in the color sensitivity of ultrasound systems affect the precision of color-flow imaging and the ability to reliably diagnose patients from all backgrounds. Systems and methods for adaptive-color imaging in ultrasound technologies are disclosed. In aspects, adaptive-color imaging techniques are used for providing sensitive color images of internal body structures. The ultrasound system can send out ultrasound waves in a transmitting process that can reflect off the internal organs or tissues of a patient. The reflected signals can be received back at the ultrasound system in a receiving process, which can then process the received signals into radio frequency (RF) data. In aspects, the RF data can be processed using a color-imaging process to extract color-flow information and filter the RF data to produce color-image data. The color-image data is data used in an ultrasound system to generate an ultrasound image.

In other aspects, feedback can be provided to adjust any one of the previously mentioned processes or processes disclosed herein. The feedback can occur through an RF-data feedback loop, a color-image-data feedback loop, or a combined RF-data and color-image-data feedback loop. The RF-data feedback loop takes weighted RF data in the form of a signal-to-noise ratio (SNR) and assigns the weighted RF data to a color box that has different regions, each with a different weight for SNR feedback. The color-image-data feedback loop is similar to the RF-data feedback loop, but instead uses a total number of pixels with color signal instead of the SNR. The combined RF-data and color-image-data feedback loop implements an adaptive process that uses one or more (including all) color-image-data properties and RF-data properties to adjust one or more parameters or filters used in any of the three processes above. The parameters can be adjusted continuously based on the feedback that is being received, thus refining each image with each iteration. The adjustments can be visualized on a user interface within the ultrasound system. Processors in the ultrasound system can also determine weighted data values for regions in an ultrasound image to evaluate cost functions used to adapt the parameters.

Unlike conventional ultrasound systems limited by color sensitivity issues, systems and methods disclosed herein improve the precision of color-flow imaging and ensure more accuracy when diagnosing patients. The continuous feedback loops optimize parameters and data properties with each iteration and provide real-time visualization of the optimization on the user interface of the ultrasound system. In aspects, the adaptability of color-imaging techniques in this document may provide users, such as clinicians and patients, with higher quality user experiences compared to conventional color-imaging ultrasound techniques.

### Example Ultrasound System

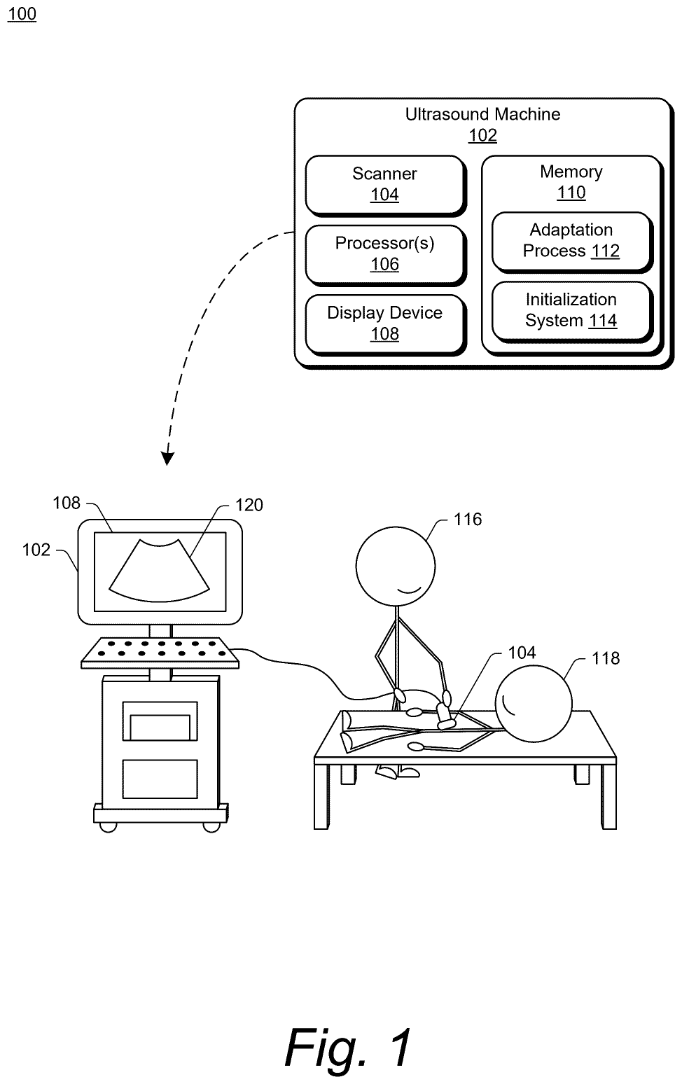

FIG. 1 illustrates an example environment for an ultrasound system 100 having an ultrasound scanner, in accordance with one or more implementations. Generally, the ultrasound system 100 includes an ultrasound machine 102, which generates data (including images) based on high-frequency sound waves reflecting off body structures. The ultrasound machine 102 includes various components, some of which include a scanner 104, one or more processors 106, a display device 108, a memory 110. In an example, the display device 108 can include multiple display devices. A first display device can display a first ultrasound image, and a second display device can display a focused ultrasound image or a segmentation image that is generated based on the first ultrasound image. In some implementations, the ultrasound machine 102 also includes an adaptation process 112 and an initialization system 114 configured to start the adaptation process 112. The adaptation process 112 can be used in accordance with one or more implementations directed at color-flow imaging.

A user 116 (e.g., nurse, ultrasound technician, operator, sonographer, etc.) directs the scanner 104 toward a patient 118 to non-invasively scan internal bodily structures (e.g., organs, tissues, etc.) of the patient 118 for testing, diagnostic, or therapeutic reasons. In some implementations, the scanner 104 includes an ultrasound transducer array and electronics coupled to the ultrasound transducer array to transmit ultrasound signals to the patient's anatomy and receive ultrasound signals reflected from the patient's anatomy. In some implementations, the scanner 104 is an ultrasound scanner, which can also be referred to as an ultrasound probe.

The display device 108 is coupled to the processor 106, which processes the reflected ultrasound signals to generate ultrasound data. The display device is configured to generate and display an ultrasound image (e.g., ultrasound image 120) of the anatomy based on the ultrasound data generated by the processor 106 from the reflected ultrasound signals detected by the scanner 104. In aspects, the ultrasound data can include data and/or the ultrasound image 120. In some embodiments, the ultrasound data (e.g., the ultrasound image 120, or data representing the ultrasound image 120) is used as input to the initialization system 114.

Based on the quality of the ultrasound data, the ultrasound system 100 generates, using at least the initialization system 114, new ultrasound data that includes parametrization for color imaging based on the adaptation process 112. For example, the initialization system 114 can input the original ultrasound data (e.g., the ultrasound image 120) into a similarity score generator, generate similarity scores, and sort the original ultrasound data with other ultrasound data in a database based on similarity scores. The initialization system 114 can select ultrasound data that corresponds to a highest similarity score, then find the parameter settings corresponding to the selected data (e.g., the data with the highest similarity score) and provide the parameter settings as an initialization to the adaptation process 112. Further details of these and other features are described below.

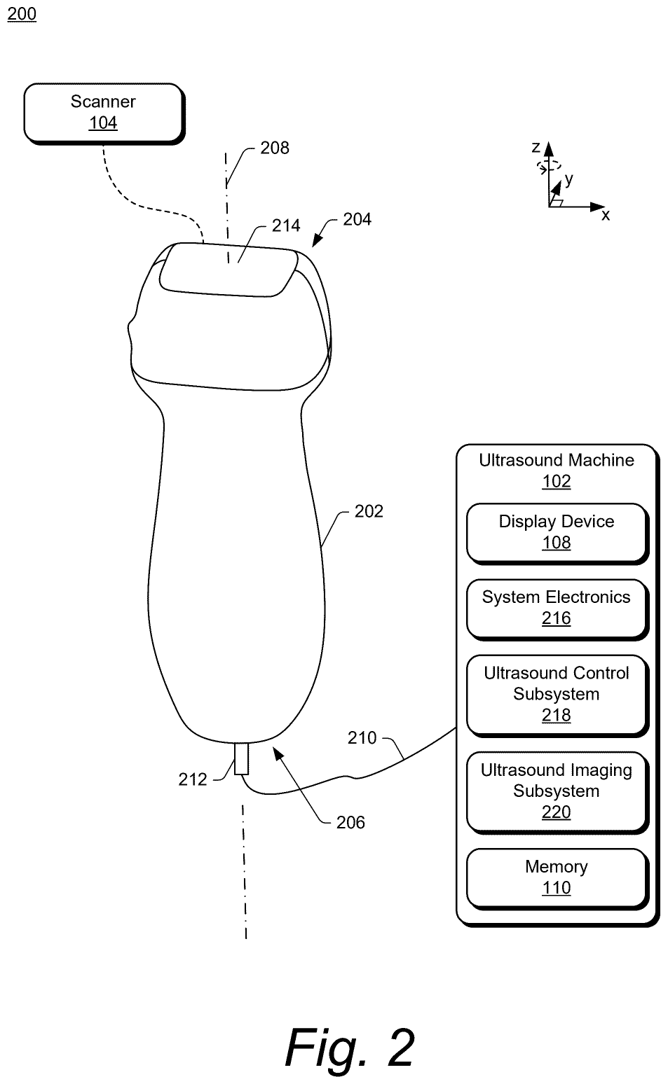

FIG. 2 illustrates an example implementation 200 of the ultrasound system 100 from FIG. 1. The scanner 104 (e.g., ultrasound scanner) includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104. In an example, the scanner 104 is electrically coupled to an ultrasound imaging system (e.g., the ultrasound machine 102) via a cable 210 that is attached to the proximal end portion 206 of the scanner 104 by a strain-relief element 212. In some implementations, the scanner 104 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 MHz) and/or high-frequency ultrasound (e.g., greater than or equal to 15 MHz). A particular frequency range to use can readily be determined based on various factors, including, for example, depth of imaging, desired resolution, and so forth. In some aspects, the transducer assembly 214 includes multiple transducer arrays, such as a first array that operates with low-frequency ultrasound and a second array that operates with high-frequency ultrasound. The first array and the second array can be operated separately or jointly. For instance, when jointly operated, harmonics of reflections of ultrasound transmitted by the first array can be received by the second array to perform tissue harmonic imaging.

In some implementations, the system electronics 216 include one or more processors (e.g., the processor(s) 106 from FIG. 1), integrated circuits, application-specific integrated circuits (ASICs), Field Programmable Gate Arrays (FPGAs), Graphics Processing Units (GPUs) and power sources to support functioning of the ultrasound machine 102. In some implementations, the ultrasound machine 102 also includes an ultrasound control subsystem 218 having one or more processors. At least one processor, FPGA, ASIC, or GPU causes electrical signals to be transmitted to the transducer(s) of the scanner 104 to emit sound waves and also receives electrical pulses from the scanner 104 that were created from the returning echoes. One or more processors, FPGAs, ASICs, or GPUs process the raw data associated with the received electrical pulses and form an image that is sent to an ultrasound imaging subsystem 220, which causes the image (e.g., the image 116 in FIG. 1) to be displayed via the display device 108. Thus, the display device 108 displays ultrasound images from the ultrasound data processed by the processor(s) of the ultrasound control subsystem 218.

In some implementations, the ultrasound machine 102 also includes one or more user input devices (e.g., a keyboard, a cursor control device, a microphone, a camera, etc.) that input data and enable taking measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In aspects, the disk storage device includes the memory 110, which is local to the ultrasound machine 102. Alternatively, the memory 110 used for storing the acquisition data can be remote, such as on a remote server communicatively connected to the ultrasound machine 102. In addition, the ultrasound machine 102 can include a printer that prints the image from the displayed data. To avoid obscuring the techniques described herein, such user input devices, disk storage device, and printer are not shown in FIG. 2.

### Color-Flow Imaging

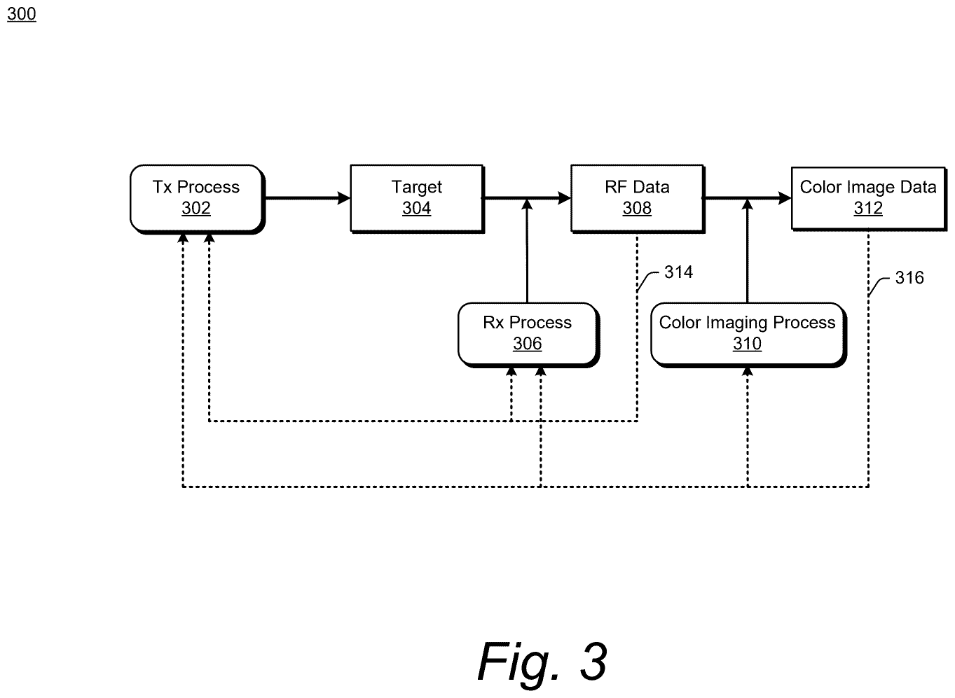

Consider FIG. 3, which illustrates an example color-flow imaging diagram 300 with a transmitting (Tx) process 302, a receiving (Rx) process 306, and a color-imaging process 310. The diagram 300 also includes a target 304, RF data 308, and color-image data 312.

The Tx process 302 includes a transmitted sequence and a transmitted waveform. The transmitted sequence includes a plurality of waveforms that are transmitted during a time window and the intervals between the transmitted waveforms. The transmitted waveform can be a composition of waveform clock numbers, delays, an aperture, a voltage, and a number of voltage levels. The transmitted sequence and waveform can be completed by a circuit, which can include a Tx and Rx switch circuit, a pulser, and a field programmable gate array (FPGA) to control the pulser. The maximum voltage is limited by the pulser as well as the safety requirements of the circuit. Transducers can transmit the waveform to the target 304.

After the waveform has been transmitted to the target 304, reflections of the waveform (e.g., reflected ultrasound signals) are received by transducers, which can be either the same transducers or different transducers from the transducers that transmitted the waveform. The received ultrasound signals pass through the Rx process 306, which includes, but is not limited to, parameters such as analog gain, analog to digital converter (ADC), delay, weight, and digital gain. Based on a variety of applications for the ultrasound signals, the above parameters can be adjusted. In some implementations, the delay and weight can be included in a beamforming function. The analog gain, ADC, and delay parameters can include filtering functions. The Rx process 306 can also include an analog front end (AFE) circuit and an FPGA circuit.

The RF data 308 and color-image data 312 are commonly used for analysis and evaluation of signals. Properties of the RF data 308 can be extracted to determine the quality of the RF data 308. The extracted data properties include, but are not limited to, data strength, noise level, center frequency, and bandwidth. Adjustments can be made to the Tx process 302 and the Rx process 306 to improve the RF data 308 based on an RF-data feedback loop 314. The improvements can be made depending on any one of the RF data 308 properties, which can be implemented through the RF-data feedback loop 314. For example, the noise level of the RF data 308 can be extracted. Based on a user input, the ultrasound system 100 can determine that the level of unwanted signals within the RF data 308 spectrum is too high, and can, in turn, adjust one or more parameters of the Tx process 302 and/or the Rx process 306 based on the RF-data feedback loop 314. The adjustments can, in turn, improve the color-image data 312.

The color-imaging process 310 is applied to the RF data 308 to further extract data properties associated with color-flow information. The color-imaging process 310 includes parameters such as a bandpass filter, a wall filter, a temporal filter, a spatial filter, and any other filter that can extract color-flow information from the RF data 308. The bandpass filter can be used to remove the carrier frequency of the RF data 308. For example, bandpass filtering can include quadrature downconversion to place the carrier frequency at or near a direct current (DC) so the carrier frequency can be removed (e.g., via filtering or subtraction of a DC offset). The wall filter can be used to remove low frequency signals within the RF data 308, which can be due to tissue motion. The wall filter can also be used to extract the color-flow information. The temporal and spatial filters can configure the color signals to reduce the color-imaging noise.

After the color-imaging process 310 is applied to the RF data 308, the color-image data 312 is produced. The color-image data 312 has multiple data properties that can be used to evaluate the quality of a color image. The color-image data 312 properties can include signal strength, color noise, frame rate, and flash artifacts. Through these properties, a color-image-data feedback loop 316 can be provided to adjust the color-imaging process 310, the Rx process 306, and/or the Tx process 302.

In some implementations, the color-image-data feedback loop 316 can operate concurrently with the RF-data feedback loop 314 on the Rx process 306 and the Tx process 302 for color optimization. In other implementations, the color-image-data feedback loop 316 can operate alone (e.g., without the RF-data feedback loop 314) on the color-imaging process 310, the Rx process 306, and the Tx process 302. In further implementations, the RF-data feedback loop 314 can operate alone (e.g., without the color-image-data feedback loop 316) on the Rx process 306 and the Tx process 302 for color optimization. Further details of these and other features are described below.

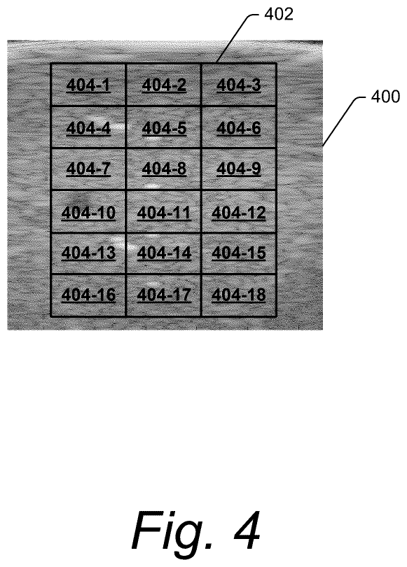

FIG. 4 illustrates an example ultrasound RF-data image 400. The image 400 is overlaid with a color box 402 displaying different regions. In aspects, the color box 402 includes weighted RF-data signal strengths and noise levels. The signal strength and noise level of the weighted RF data can be used to provide feedback to adjust the transmitted waveform in the Tx process 302, and the analog gain and digital gain in the Rx process 306. The signal strength and noise level are represented in a signal-to-noise ratio (SNR) of the weighted RF data. In color imaging, the spatial resolution requirement (e.g., the ability of an imaging system to capture and distinguish between details in an image) is typically lower than in other imaging (e.g., B-mode imaging), thus there is more room for improvement in RF-data signal strength.

In FIG. 4, the color box 402 has 18 individual regions 404 (404-1, 404-2, 404-3, 404-4, . . . , 404-18), where each region 404 has different weighted SNR data that is used to provide feedback. In aspects, the objective for analyzing the RF-data image 400 is to maximize the weighted SNR data in the color box 402. An ultrasound system (e.g., ultrasound system 100) can adapt one or more of the parameters used in the Tx and/or Rx processes to maximize the weighted SNR data within the color box 402. For example, the color box 402 can have 18 individual regions 404 as illustrated in FIG. 4. Each region 404 also has its own RF data from which the SNR is calculated. In Equation (1) below, the total weighted SNR data, SNRₜₒₜₐₗ, can be expressed as:

$${SNR}_{total} = {\sum }_{i} {\sum }_{j} {w}_{ij} {SNR}_{ij} \qquad Eq . ( 1 )$$

where i and j are the row and column indexes of the individual regions 404 from the color box 402, wᵢⱼ is the weight of a corresponding region, and SNRᵢⱼ is the averaged SNR data (calculated from the RF data) of the corresponding region. In FIG. 4, there are three columns and six rows in total, thus the regions 404-1, 404-2, 404-3, 404-4, 404-5, 404-6, 404-7, 404-8, 404-9, 404-10, 404-11, 404-12, 404-13, 404-14, 404-15, 404-16, 404-17, and 404-18 can have weights w₁₁, w₁₂, w₁₃, w₂₁, w₂₂, w₂₃, w₃₁, w₃₂, w₃₃, w₄₁, w₄₂, w₄₃, w₅₁, w₅₂, w₅₃, w₆₁, w₆₂, and w₆₃, respectively. In aspects, the total sum of the weights is equal to one, and the weights are real numbers between zero and one, as expressed in Equation (2):

$${\sum }_{i} {\sum }_{j} {w}_{ij} = 1 \qquad Eq . ( 2 )$$

Thus, the ultrasound system can determine Tx and Rx parameters that maximize Equation (1), subject to the constraint on the weights in Equation (2), meaning that the weights are real numbers between zero and one and have a sum equal to one. The weights can be determined in any appropriate way, and then normalized to satisfy the constraint in Equation (2).

For example, the weights can be calculated based on the reasonable assumption that the color box 402 surrounds a region of interest. Because the center of the color box 402 is likely where the region of interest is most prevalent, the weight is higher at points or areas closer to the center of the color box 402. Thus, as an example, w₁₁, w₁₂, w₁₃, w₂₁, w₂₂, w₂₃, w₃₁, w₃₂, w₃₃, w₄₁, w₄₂, w₄₃, w₅₁, w₅₂, w₅₃, w₆₁, w₆₂, and w₆₃ can be 0.02, 0.02, 0.02, 0.04, 0.08, 0.04, 0.08, 0.12, 0.08,0.08, 0.12, 0.08, 0.04, 0.08, 0.04, 0.02, 0.02, and 0.02, respectively. The sum of all the weights in the color box 402 is equal to one, and the center 404-8 of the color box 402 has the highest weight.

In another example, the weights can be determined to maximize SNRₜₒₜₐₗ from Equation (1). So, the weights can be determined according to Equation (2A):

$${w}_{ij}^{*} = {arg max}_{{w}_{ij}} \{ {SNR}_{total} \} \qquad Eq . ( 2 A )$$

Note that, again, the weight determinations are subject to the constraints on the weights of Equation (2), or any other suitable constraint on the weights, their sum, and/or their magnitudes.

In another example, instead of constraining the weights to Equation (2) and maximizing the SNRₜₒₜₐₗ in Equation (1), a cost function J₁ can be used. The cost function J₁ combines the SNRₜₒₜₐₗ with a tunable and soft constraint (e.g., a variable) calculated from the difference of unity and the sum of the weights. This is expressed in Equation (2B):

$${J}_{1} = ∝ \cdot {SNR}_{total} + β \cdot \vert 1 - {\sum }_{i} {\sum }_{j} {w}_{ij} \vert \qquad Eq . ( 2 B )$$

where ∝ and β are positive, real-valued scalars that can be selected to tune the relative costs of the total SNR, with the sum of the weights being different from unity. In this example, the weights can be selected in any suitable way, (e.g., according to Equation (2A) or Equation (2C) below), and then used to evaluate the cost J₁ in Equation (2B).

In some implementations, the objective is to balance the SNR data across different regions of the RF data and eventually achieve higher-quality color signals across the entire color box 402, instead of pursuing the maximum total SNR inside the color box 402. A threshold of the SNR data is preset to maintain a minimum SNR value for reasonable calculations, such as 6 decibels (dB), before a total SNR value can be calculated and maintained. For any RF data value with an SNR higher than 6 dB, a smaller weight can be used when determining the weights. Thus, the higher the SNR value, the smaller the weight of the RF data. Using this approach, the weights of the RF data can be determined with Equation (2C):

$${w}_{ij} = \frac{\partial }{⌈ {SNR}_{ij} - T ⌉} \qquad Eq . ( 2 C )$$

where T is a threshold on the SNR (e.g., 6 dB), ∂ is a real-valued, positive scalar for setting the proportionality scale, and the operator ┌(·)┐=(·) if (·)≥0, and Δ if (·)<0, for a real-valued, positive scalar Δ. For instance, Δ can be used to set the weights for regions that have an SNR value less than the threshold T. In an example, Δ can be set to a small number, giving higher weights to the SNR's that do not satisfy the threshold. In another example, Δ can be set to a large number, effectively zeroing the weights of the SNR values that do not satisfy the threshold, so that these SNR values do not contribute to the total SNR or to the cost function of the total SNR used to adjust the Tx and/or Rx parameters.

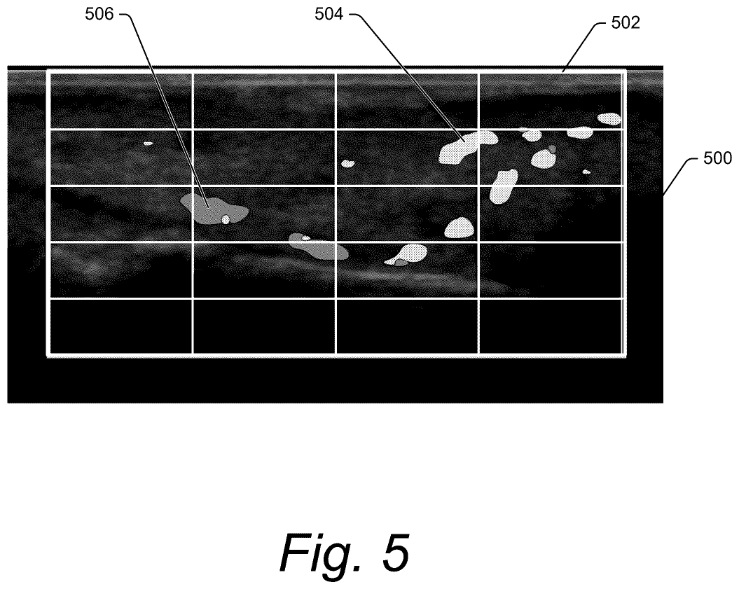

FIG. 5 illustrates an example color-image-data image 500. The image 500 is overlaid with a color box 502 displaying different regions, similar to the color box 402. The image 500 is illustrated with dotted areas representing different colors. The different colors (or dot-densities) represent blood flow in different directions. Within the color box 502, areas 504 are overlaid with a first color by the ultrasound system to represent blood flow in a first direction and areas 506 are overlaid with a second, different color to represent blood flow in a second, opposite direction compared to the first direction.

Instead of RF data, color-image data is used to provide feedback to adjust the color-imaging process. To calculate the total strength of the color-image data, a weighted color signal strength by distance to the center can be used. In this example, the total SNR value is not calculated. Instead, the total number of pixels with color signals above a preset threshold is calculated with Equation (3):

$${Pixel}_{total} = {\sum }_{i} {\sum }_{j} {w}_{ij} {Pixel}_{ij} \qquad Eq . ( 3 )$$

where i and j are the row and column indexes of the individual regions from the color box 502, wᵢⱼ is the weight of the corresponding region, and Pixelᵢⱼ is the pixel number percentage of the region (e.g., the percentage of pixels in the region having color signals above the preset threshold). In FIG. 5, there are four columns and five rows in total, making 20 individual regions. The total sum of the weights is equal to one. For example, the weights can be uniform (e.g., a value of 0.05) or the center regions can have higher weights, while the outside regions can have lower weights. In some implementations, the weights can be determined in a similar fashion as Equation (2C), except with SNRᵢⱼ being replaced by Pixelᵢⱼ.

In aspects, the weights can be determined based on the color signal strength (e.g., color power) alone. The objective is to balance the color signal strength across different regions of the color-image-data image 500 and to eventually achieve higher-quality color signals across the entire color box 502. A threshold of color signal strength (e.g., 6 dB) may be preset to maintain a minimum color signal strength. In some instances, a total SNR (or cost function) can still be calculated and maintained. For color signals with a strength higher than, for example, a threshold (e.g., 6 dB), the weight is smaller. In other implementations, the weights can be determined in a similar fashion as Equation (2C), except with SNRᵢⱼ being replaced by color signal strength for the region.

In other aspects, the weights can be determined based on the color-flow velocity. The ultrasound system (e.g., ultrasound system 100) is typically more sensitive to high-velocity flows and less sensitive to low-velocity flows. The objective is to increase the sensitivity to the low-velocity flows across different regions of the color-image-data image 500. A threshold of color signal strength (e.g., 6 dB) may be preset to maintain a minimum color signal strength. The total number of pixels with the color signal above the preset threshold can be calculated similar to Equation (3). For example, the weights can be determined by using the inverse of the color-flow velocity value.

In some implementations, frame rate is used as a factor to provide feedback. In general, different applications can require different frame rates. For example, a cardiac examination can require a higher frame rate than other examinations because the heart is a continually moving organ that has rapidly moving parts, therefore the higher frame rate can more clearly capture these essential movements. Thus, if a minimum frame rate is set as a threshold, these parameters can be adjusted in the Tx process and/or the color-imaging process to reach higher color sensitivity.

In other implementations, flash artifact is used as a factor to provide feedback. Because of slow tissue motion, there may be flash artifacts in color images taken with the ultrasound system that can distort the color-image data. The objective is to suppress the flash artifacts without impacting the real color signals. For example, the global flash artifacts can be calculated, and different weights can be applied with respect to the real color signal regions within the color box 502. A relatively higher weight can be applied to regions that are closer to the real color signals to suppress the flash artifacts.

In aspects, the Tx process 302, the Rx process 306, and the color-imaging process 310 are adjusted adaptively based on the feedback from the color-image data 312. One or more (including all) of the color-image data 312 properties (e.g., color signal strength, color noise, frame rate, and flash artifacts level) can be used as feedback to adjust one or more of the parameters in the color-imaging process 310 (e.g., bandpass filter, wall filter, temporal filter, and spatial filter), the Rx process 306 (e.g., analog gain, ADC, delay, weight, and digital gain), and the Tx process 302 (e.g., Tx sequence and Tx waveform).

In other aspects the Tx process 302, the Rx process 306, and the color-imaging process 310 are adjusted adaptively based on the feedback from the color-image data 312 and the RF data 308. One or more (including all) of the color-image data 312 properties (e.g., color signal strength, color noise, frame rate, and flash artifacts level) and one or more (including all) of the RF data 308 properties (e.g., data strength, noise level, center frequency, and bandwidth) can be used as feedback to adjust one or more of the parameters in the color-imaging process 310 (e.g., bandpass filter, wall filter, temporal filter, and spatial filter), the Rx process 306 (e.g., analog gain, ADC, delay, weight, and digital gain), and the Tx process 302 (e.g., Tx sequence and Tx waveform).

The ultrasound system 100 can adjust one or more of the parameters in the color-imaging process 310, the Rx process 306, and/or the Tx process 302 to increase (e.g., maximize) a total SNR (e.g., SNRₜₒₜₐₗ), or a cost function of the total SNR, as previously described. For example, the ultrasound system determines a value of a first parameter that optimizes the cost function, fixes the first parameter value, and then begins optimizing the cost function over a second parameter, using the value of the first parameter previously determined. Once the value of the second parameter is determined, the system can use the values for the first and second parameters to determine a value for a third parameter that optimizes the cost function. In this way, the system can sequentially (e.g., one at a time), determine the optimum parameter settings for the color-imaging process 310, the Rx process 306, and/or the Tx process 302.

In another example, the ultrasound system 100 can, at each optimization step or iteration, simultaneously adjust two or more parameters of one or more of the color-imaging process 310, the Rx process 306, and the Tx process 302, and evaluate a cost function as previously described. For example, the parameters can be aggregated into a vector, and the ultrasound system 100 can update the vector via a stochastic gradient ascent over the vector of a cost function as previously described. For instance, the parameter values of the vector f can be determined at each discrete iteration n according to Equation (4):

$${f}_{n + 1} = {f}_{n} + μ \cdot \frac{δ J}{δ f} \qquad Eq . ( 4 )$$

for a step size (small number) μ. The gradient can be numerically determined by calculation of the change in the total SNR (or cost function of the total SNR) resulting from the change in the parameter vector. Additionally or alternatively, the ultrasound system can determine the optimum value of parameters in the vector by searching over a finite number of choices of the parameterization and selecting the parameters with the lowest cost. Additionally or alternatively, the system can use a binary search to determine the parameterization, by searching over ranked values of a given parameter aggregated into a vector and selecting the values based on a relative increase or decrease of the result of the cost function. By evaluating the cost function over multiple values of parameters of the color-imaging process 310, the Rx process 306, and/or the Tx process 302, the ultrasound system 100 can determine a parameter configuration for optimized color imaging.

The convergent properties and settings provided by the adaptation process can be sensitive to the initialization of the parameters. If poorly initialized, the adaptation process can diverge and not generate a parameterization that is useful for improving the color imaging. Using the techniques disclosed herein, the ultrasound system 100 can generate an initialization of the parameters of the color-imaging process 310, the Rx process 306, and/or the Tx process 302 that can be used by an adaptation process (e.g., adaptation process 112), thereby improving the results of the adaptation process.

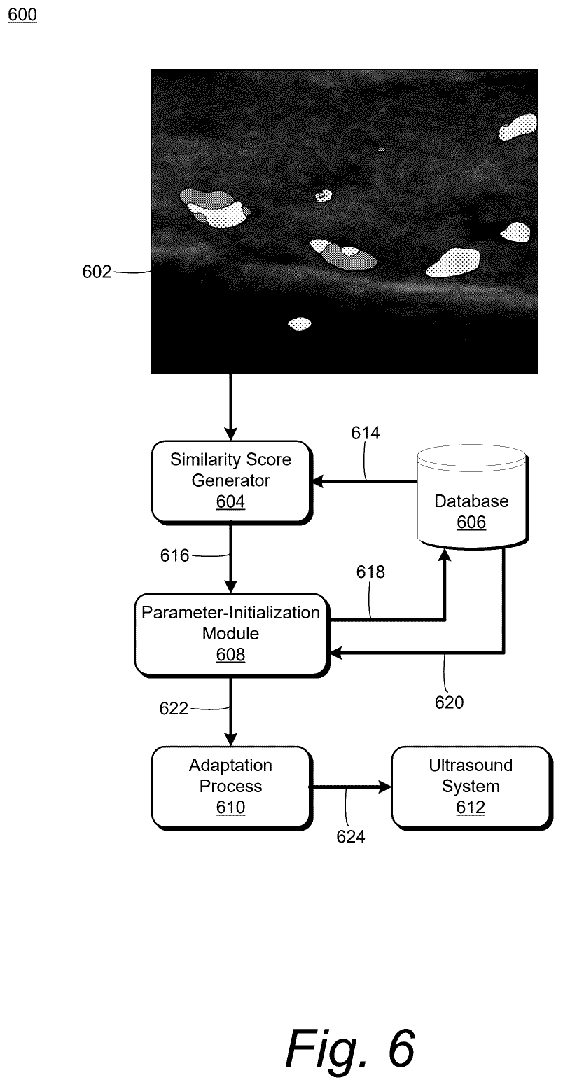

FIG. 6 illustrates an example initialization system 600 for an adaptation process. The initialization system 600 provides an ultrasound image 602 to a similarity score generator 604. The similarity score generator 604 receives images 614 from a database 606 that maintains the images 614 and parameter settings associated with the images 614. For instance, the database 606 can store pairs of images (e.g., a “before image” and an “after image”) and the parameter settings determined for the pair (e.g., parameters for the “before image” used to generate the “after image”). The similarity score generator 604 generates similarity scores 616 that represent similarities between the ultrasound image 602 and the images 614 provided by the database 606. The similarity score generator 604 can implement any suitable function to generate the similarity scores 616. For example, the similarity score generator 604 can implement a machine-learned model (e.g., a neural network) to generate the similarity scores 616. Additionally or alternatively, the similarity score generator 604 can implement a stochastic or deterministic signal processing model to generate the similarity scores 616. The similarity score generator 604 provides the similarity scores 616 to a parameter-initialization module 608.

The parameter-initialization module 608 ranks the similarity scores 616 from the similarity score generator 604 and selects an image from the images 614 based on the ranking. For example, the parameter-initialization module 608 can select an image that corresponds to a highest similarity score. The parameter-initialization module 608 then sends a parameter request 618 to the database 606 for parameter settings 620 corresponding to the selected image (e.g., the image with the highest similarity score). The database 606 provides the parameter settings 620 back to the parameter-initialization module 608, which in turn provides the parameter settings 620 as a parameter initialization 622 to the adaptation process 610. For example, the initialization system 600 uses the parameter settings 620 directly from the database 606 to configure an ultrasound system 612 for color imaging, without further adaptation by the adaptation process 610. Alternatively, the adaptation process 610 can use the parameter settings 620 as parameter initializations 622 and adapt the parameter settings 620 via one of the adaptation processes and by using one of the cost functions as described above. The adaptation process 610 can then provide a parameterization 624 determined from the adaptation process 610 to the ultrasound system 612 for color imaging.

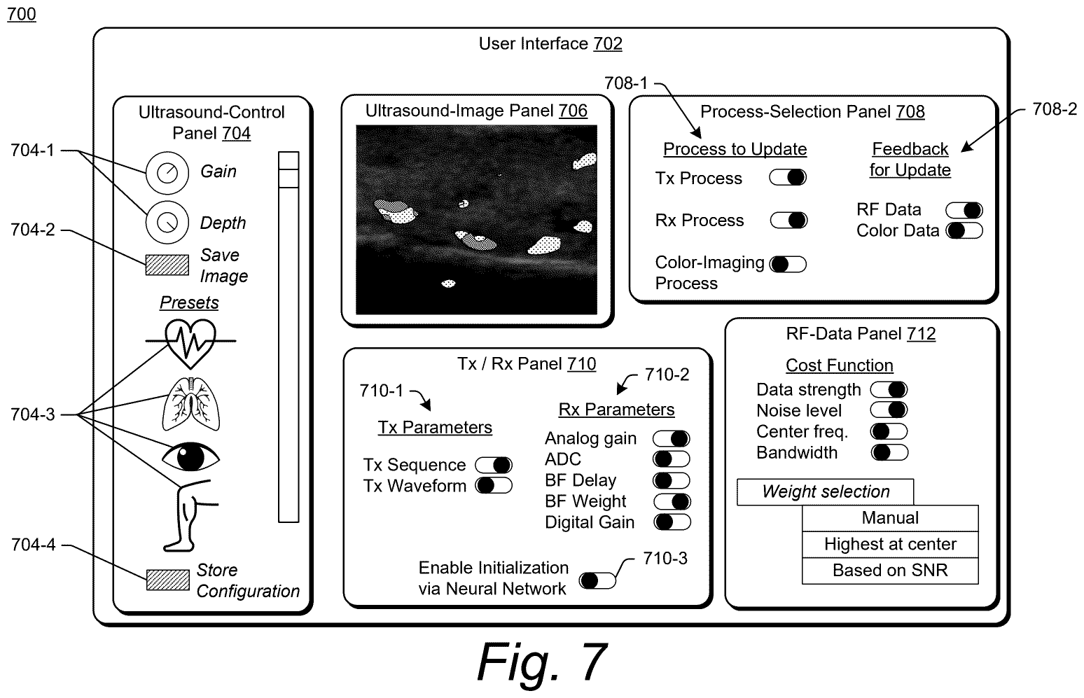

FIG. 7 illustrates an example implementation 700 of a display of a user interface 702 associated with an ultrasound system (e.g., ultrasound system 100) in accordance with one or more implementations. In aspects, the user interface 702 can be rendered via the display device 108. The user interface 702 includes an ultrasound-control panel 704, which includes various controls, examples of which include gain and depth adjustment controls 704-1, an image-saving control 704-2, presets 704-3 for specific examination types, and a color-optimized configuration-storage control 704-4. The examination presets 704-3 are represented by selectable icons for various examinations, examples of which include a cardiac examination, a respiratory examination, an ocular examination, and a muscular-skeletal examination. These examination presets 704-3, when selected, can configure the ultrasound machine with predetermined values of gain, depth, and other imaging parameters (e.g., beamformer settings and transducer frequencies). For example, one or more parameters set through an examination preset 704-3 can be used as initialization values for an adaptation process (e.g., adaptation process 610) that adapts parameters of the color-imaging process 310, the Rx process 306, and/or the Tx process 302 for color imaging, as previously described. The color-optimized configuration-storage control 704-4 can be used to store the parameters after the adaptation process in a color-optimized configuration (e.g., as a preset) so that the parameters can be quickly retrieved and used in a subsequent ultrasound examination.

The user interface 702 also includes an ultrasound-image panel 706 for displaying any suitable type and/or number of ultrasound images (e.g., ultrasound image 120). A process-selection panel 708 can also be included within the user interface 702. The process-selection panel 708 enables selection of one or more processes to update 708-1 (e.g., Tx process, Rx process, color-imaging process) and selection of the feedback for update 708-2 (e.g., the type of data for adapting the parameters). For example, as illustrated in FIG. 7, the selections in the process-selection panel 708 indicate that the Tx process and Rx process parameters are to be adapted based on the RF data. In response to these selections, the user interface 702 can provide a Tx/Rx panel 710 and an RF-data panel 712. The Tx/Rx panel 710 includes selections for adaptable parameters of the Tx process and the Rx process (e.g., Tx parameters 710-1 and Rx parameters 710-2). In this example, the selections indicate that parameters of the Tx sequence should be adapted, and that for the Rx process, parameters including the analog gain and beamformer (BF) weights should be adapted for color imaging. To adapt these parameters, the Tx/Rx panel 710 can include a selectable option 710-3 for their initialization via a neural network (e.g., as described with respect to FIG. 6).

As a response to the selection of RF data for use as the feedback for update 708-2 in the process-selection panel 708, the user interface 702 displays the RF-data panel 712. The RF-data panel 712 can include selections for a cost function that is to be optimized (e.g., maximized) to adapt the parameters selected in the Tx/Rx panel 710. The selections in the example in the RF-data panel 712 indicate that RF-data strength and noise level are to be used in a cost function for the optimization. For instance, a cost function described above with respect to FIG. 4 that uses a weighted SNR in a user-selected color box 402 can be used based on the selections in the RF-data panel 712. The RF-data panel 712 can also include a pull-down tab for user selection of the weights used in the cost function. The user interface 702 can receive a user input, via the RF-data panel 712, that enters the weight values, sets the weights with the highest weight at the center, and/or sets the weights based on the total SNR. The user interface 702 can also include a menu option (not illustrated) to select the number of weights to be used (e.g., the number of regions in a color box).

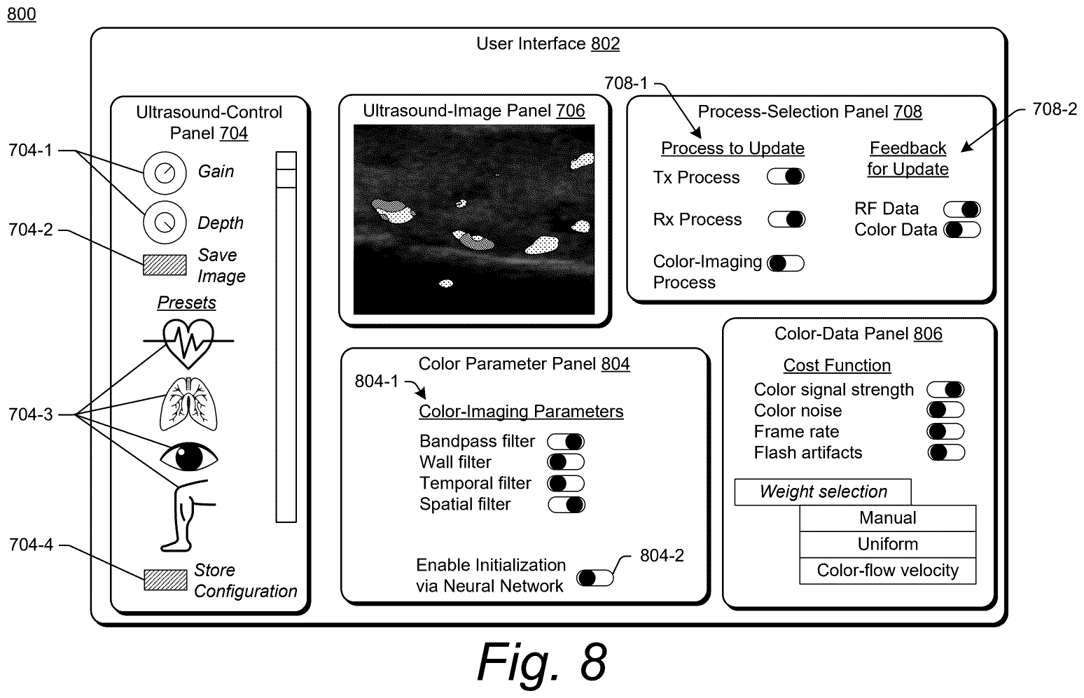

FIG. 8 illustrates another example implementation 800 of a display of a user interface 802 associated with an ultrasound system (e.g., ultrasound system 100) in accordance with one or more implementations. In aspects, the user interface 802 can be rendered via the display device 108. The user interface 802 is illustrated with the ultrasound-control panel 704, the ultrasound-image panel 706, and the process-selection panel 708 from FIG. 7. In contrast to the user interface 702, in the user interface 802 the process-selection panel 708 includes selections indicating that the color-imaging process parameters are to be adapted based on color data. In response to these selections, the user interface 802 provides a color parameter panel 804 and a color-data panel 806.

The color parameter panel 804 includes selections for adaptable parameters 804-1 of the color-imaging process. The adaptable parameters 804-1 can include a bandpass filter, a wall filter, a temporal filter, a spatial filter, etc. As illustrated in FIG. 8, the selections indicate that parameters of the bandpass filter and spatial filter should be adapted for color imaging. To adapt these parameters 804-1, the color parameter panel 804 includes a selectable option 804-2 for their initialization via a neural network (e.g., as described with respect to FIG. 6).

In response to selecting the color data for use as the feedback for update 708-2 in the process-selection panel 708, the user interface 802 displays the color-data panel 806. The color-data panel 806 includes selections for a cost function that is to be optimized (e.g., maximized) to adapt the parameters 804-1 selected in the color parameter panel 804. The selections in the color-data panel 806 indicate that color signal strength is to be used in a cost function for the optimization. For instance, a cost function described above with respect to FIG. 5 that uses a weighted color signal in a user-selected color box 502 can be used based on the selections in the color-data panel 806. The color-data panel 806 can also include a pull-down tab for user selection of the weights used in the cost function. The user interface 802 can receive a user input, via the color-data panel 806 that enters the weight values, sets the weights uniformly, and/or sets the weights based on the color-flow velocity (e.g., inversely proportional to the color-flow velocity, as previously described).

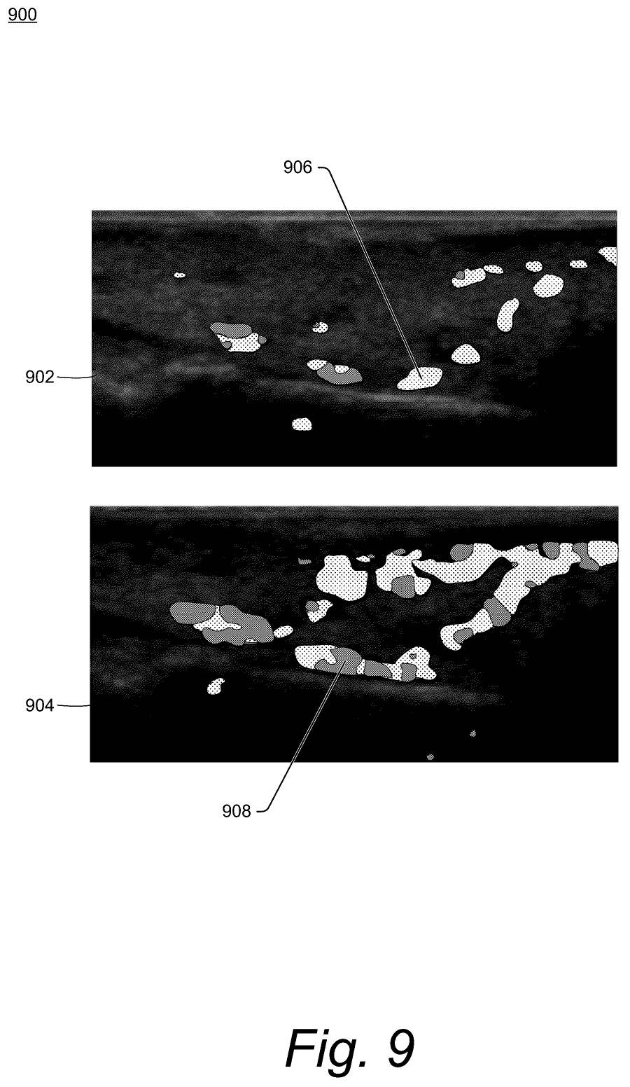

In response to the adaptation process being initiated through the initialization system in the ultrasound system (e.g., through an “adapt now” button, not illustrated), the ultrasound-image panel 706 can display an ultrasound image (e.g., ultrasound image 120, ultrasound image 602) generated based on the parameters that have been adapted. For example, the ultrasound-image panel 706 can display multiple images, one image generated with parameters before the adaptation, and another image generated with parameters that have been adapted (described in further detail in FIG. 9). For example, a user can select the number of images that are to be displayed in the ultrasound-image panel 706, and the system can display a corresponding number of ultrasound images, including multiple ultrasound images that have been generated based on different parameterizations achieved via the adaptation process. For example, one ultrasound image can correspond to the adaptation of parameters in the Tx and Rx processes, another ultrasound image can correspond to the adaptation of parameters in the color-imaging process, and still another ultrasound image can correspond to the adaptation of parameters in Tx, Rx, and color-imaging processes.

FIG. 9 illustrates an example implementation 900 of a before-adaptation image 902 and an after-adaptation image 904 associated with an ultrasound system. The before-adaptation image 902 is generated with parameters before an adaptation process was initiated, and the after-adaptation image 904 is generated with parameters that have been adapted. The before-adaptation image 902 and the after-adaptation image 904 are illustrated with dotted areas representing different colors. The different colors (or dot-densities) represent blood flow in different directions. Within both the before-adaptation image 902 and the after-adaptation image 904, areas 906 are overlaid with a first color by the ultrasound system to represent blood flow in a first direction and areas 908 are overlaid with a second, different color to represent blood flow in a second, opposite direction compared to the first direction.

In the example of FIG. 9, the before-adaptation image 902 and the after-adaptation image 904 are generated based on color signal strength being the selected property of the color data to adjust the Tx process and the color-imaging process. After iteratively adapting the Tx process and the color-imaging process, a much more sensitive color image can be produced, as illustrated by the after-adaptation image 904. In aspects, the before-adaptation image 902 and after-adaptation image 904 can be displayed via a display device (e.g., display device 108) or in an ultrasound-image panel (e.g., ultrasound-image panel 706).

### Example Methods

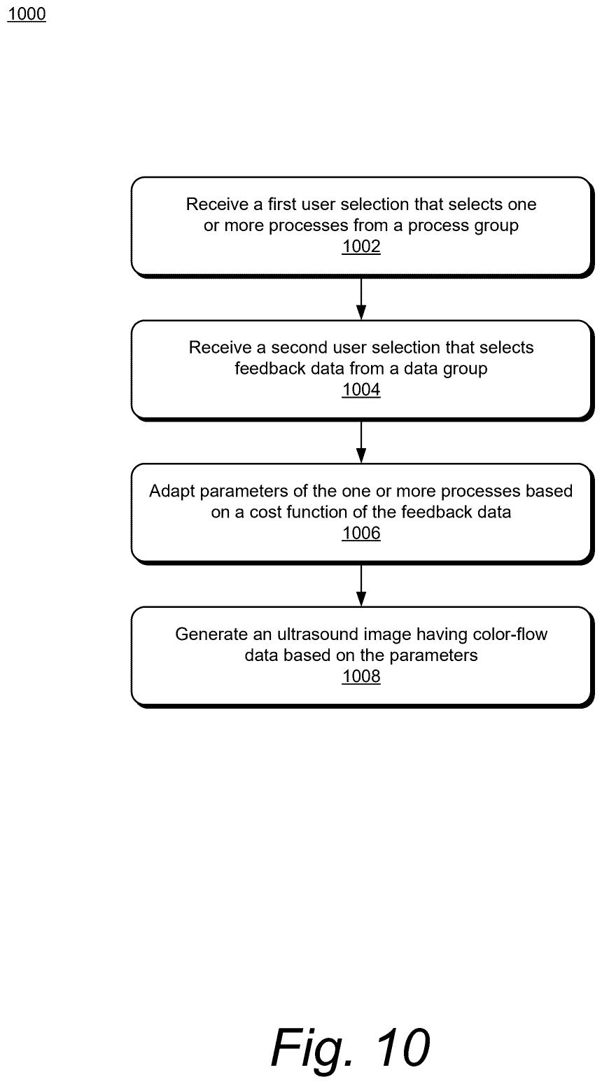

FIGS. 10-12 depict methods 1000, 1100, and 1200, respectively for generating optimized ultrasound images. The methods 1000, 1100, and 1200 are shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example system 100 of FIG. 1 or to entities or processes as detailed in FIGS. 2-9, reference to which is made for example only. The techniques are not limited to performance by one entity or multiple entities operating on one device.

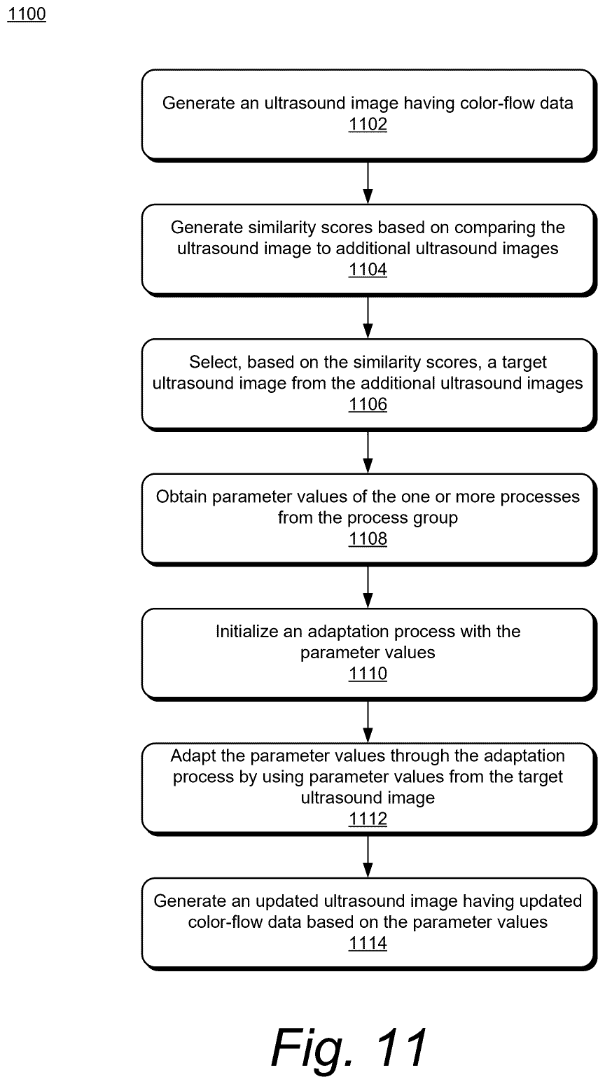

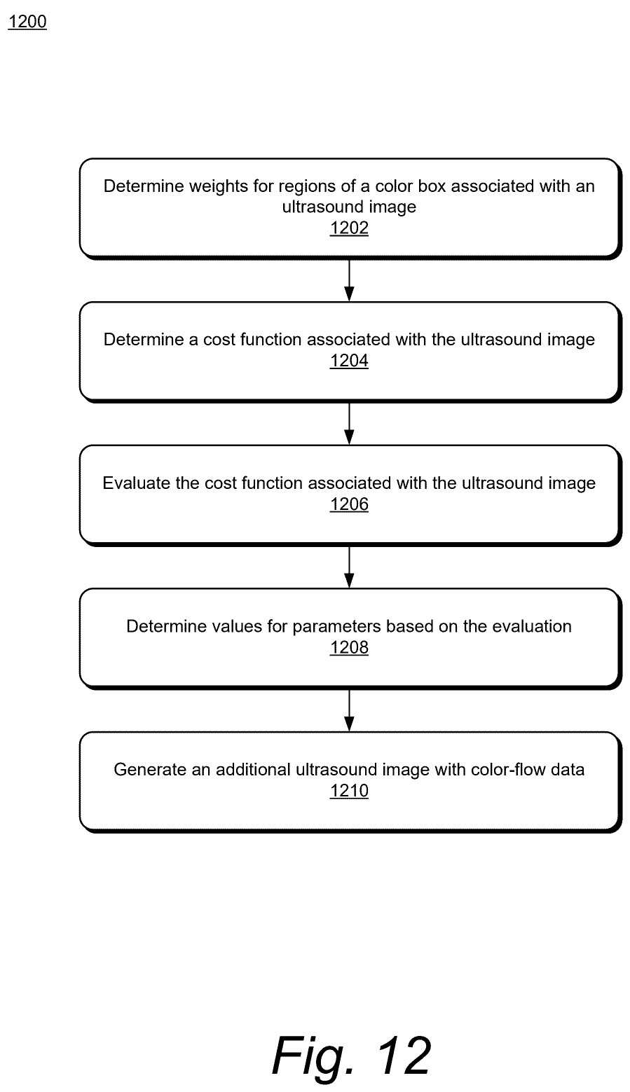

FIG. 10 depicts a method for generating ultrasound images based on adapted parameters. FIG. 11 depicts another method for generating ultrasound images based on adapted parameters. FIG. 12 depicts a method for generating ultrasound images based on a cost function of selected feedback data. The methods 1000, 1100, and 1200 can be performed by the ultrasound system 100, including the ultrasound machine 102.

At 1002 in FIG. 10, a first user selection is received that selects one or more processes from a process group. The process group includes the Tx process 302, the Rx process 306, and the color-imaging process 310. In an example, the first user selection can be the selection of the Tx process and the Rx process in the process-selection panel 708 of the user interface 702. In another example, the first user selection can be the selection of the color-imaging process in the process-selection panel 708 of the user interface 802.

At 1004, a second user selection is received that selects feedback data from a data group. The data group includes the RF data 308 and the color-image data 312. In an example, the second user selection can be the selection of the RF data in the process-selection panel 708 of the user interface 702. In another example, the second user selection can be the selection of the color data in the process-selection panel 708 of the user interface 802.

At 1006, parameters of the one or more processes are adapted based on a cost function of the feedback data. For example, as illustrated in the Tx/Rx panel 710 of FIG. 7, the Tx sequence of the Tx parameters 710-1 and the analog gain and BF weight of the Rx parameters 710-2 are selected to be adapted. The cost function is associated with the selected RF data and can be evaluated in different ways. For example, the cost function can be evaluated based on an SNR of the RF data.

At 1008, an ultrasound image having color-flow data is generated based on the parameters. For example, the after-adaptation image 904 can be displayed in the ultrasound-image panel 706 when the parameters have been adapted.

In some implementations, the method 1100 in FIG. 11 can continue from the method 1000 in FIG. 10. In other implementations, the method 1100 can begin independent of the method 1000. In FIG. 11 at 1102, an ultrasound image with color-flow data is generated. For example, the user 116 can use the scanner 104 of the ultrasound machine 102 to generate the ultrasound image 120 for display via the display device 108. In some examples, the ultrasound image with color-flow data can be the after-adaptation image 904.

At 1104, similarity scores are generated based on comparing the ultrasound image with additional ultrasound images. For example, in FIG. 6, the similarity score generator 604 receives images 614 from a database 606 that maintains images 614 and parameter settings associated with the images 614. The database 406 can store pairs of images (e.g., a “before image” and an “after image”) and the parameter settings determined for the pair (e.g., parameters for the “before image” used to generate the “after image”). The similarity score generator 604 generates similarity scores 616 that represent similarities between the ultrasound image 602 and the images 614 provided by the database 606. The similarity score generator 604 can implement any suitable function to generate the similarity scores 616. For example, the similarity score generator 604 can implement a machine-learned model (e.g., a neural network) to generate the similarity scores 616. Additionally, or alternatively, the similarity score generator 604 can implement a stochastic or deterministic signal processing model to generate the similarity scores 616.

At 1106, a target ultrasound image is selected from the additional ultrasound images based on the similarity scores. For example, in FIG. 6, the parameter-initialization module 608 ranks the similarity scores 616 from the similarity score generator 604 and selects a target ultrasound image from the images 614 based on the ranking of the similarity score 616. The parameter-initialization module 608 can select a target ultrasound image that corresponds to the highest similarity score to the images 614.

At 1108, parameter values of the one or more processes from the process group are obtained. As illustrated in FIG. 6, the parameter-initialization module 608 sends a parameter request 618 to the database 606 for parameter settings 620 corresponding to the target ultrasound image. The database 606 provides the parameter settings 620 back to the parameter-initialization module 608.

At 1110, an adaptation process is initialized with the parameter values. In FIG. 6, the parameter-initialization module 608 provides the parameter settings 620 as a parameter initialization 622 to the adaptation process 610. In FIG. 7, the Tx/Rx panel 710 includes a selection 710-3 to enable initialization via a neural network, which begins the adaptation process of the parameters.

At 1112, the parameter values are adapted through the adaptation process by using parameter values from the target ultrasound image. For example, the adaptation process 610 can use the parameter settings 620 as parameter initializations 622 and adapt the parameter settings 620 via one of the adaptation processes (e.g., a preset process). The adaptation process 610 can then provide the parameterization 624 determined from the adaptation process 610 to the ultrasound system 612 for color imaging.

At 1114, an updated ultrasound image with updated color-flow data is generated based on the parameter values. The parameterization 624 is provided to the ultrasound system 612 (e.g., ultrasound system 100) which can generate a new ultrasound image via the display device (e.g., display device 108).

At 1202 in FIG. 12, weights for regions of a color box associated with an ultrasound image are determined. The weights can be determined in different ways. For example, the weights can be determined through maximizing the total SNR value (e.g., a cost function of the total SNR) over the color box in the ultrasound image and finding the average SNR value for each region based on the corresponding RF data. In another example, the weights can also be determined based on the reasonable assumption that the color box surrounds a region of interest, so the center of the color box is likely where the region of interest is most prevalent and will therefore have a higher weight. In a further example, the weights can be determined through maximizing and balancing the total color signal strength over the color box in the ultrasound image and calculating a uniform color signal strength value for each region based on the corresponding color-image data. In another example, the weights can be determined by using the inverse of the color-flow velocity in the ultrasound image. Further, the weights can be determined through Equations (2A) or (2C).

At 1204, a cost function associated with the ultrasound image is determined. The cost function is determined based on ultrasound data associated with the ultrasound image or based on color-image data generated from the ultrasound data. The cost function can be determined in different ways. For example, the cost function can be determined through selecting a hard constraint for the weights in a color box region. The hard constraint can be that the weights must be values between zero and one or that the total sum of the weights is equal to one, as expressed in Equation (2). As another example, the cost function can be determined through selecting a soft constraint for the weights in a color region. The soft constraint can be a variable calculated from the difference of unity and the sum of the weights, as expressed in Equation (2B).

At 1206, the cost function associated with the ultrasound image is evaluated. The cost function is evaluated over multiple values of parameters associated with the process group. The cost function can be evaluated in different ways. For example, the cost function can be evaluated by using Equation (2B). In another example, the cost function can be evaluated through the initialization system 600. The initialization system 600 provides an ultrasound image 602 (e.g., the ultrasound image) to a similarity score generator 604. The similarity score generator 604 receives images 614 from a database 606 that maintains the images 614 and parameter settings associated with the images 614. The similarity score generator 604 generates similarity scores 616 (e.g., cost functions) that represent similarities between the ultrasound image 602 and the images 614. The similarity score generator 604 can implement any suitable function to generate the similarity scores 616. For example, the similarity score generator 604 can implement a machine-learned model (e.g., a neural network) to generate the similarity scores 616. Additionally or alternatively, the similarity score generator 604 can implement a stochastic or deterministic signal processing model to generate the similarity scores 616. The similarity score generator 604 provides the similarity scores 616 to a parameter-initialization module 608. The parameter-initialization module 608 ranks the similarity scores 616 from the similarity score generator 604 and selects an image from the images 614 based on the ranking. For example, the parameter-initialization module 608 can select an image that corresponds to a highest similarity score (e.g., a maximized cost function).

At 1208, based on the evaluation, values for parameters are determined. The parameter values are associated with the ultrasound data of the ultrasound image. The values can be determined through enabling an initialization system, such as the initialization system 600. The parameter-initialization module 608 in the initialization system 600 sends a parameter request 618 to the database 606 for parameter settings 620 corresponding to an ultrasound image (e.g., the image with the highest/lowest cost). The database 606 provides the parameter settings 620 back to the parameter-initialization module 608, which in turn provides the parameter settings 620 as a parameter initialization 622 to the adaptation process 610. For example, the initialization system 600 can obtain the parameter settings 620 directly from the database 606 to configure an ultrasound system 612 for color imaging, without further adaptation by the adaptation process 610. Alternatively, the adaptation process 610 can use the parameter settings 620 as parameter initializations 622 and adapt the parameter settings 620 via one of the adaptation processes and by using one of the cost functions as described above.

The values can also be determined through maximizing the evaluated cost function. For example, the ultrasound system can determine a value of a first parameter that maximizes the evaluated cost function, fix the first parameter value, and then determine a second parameter that maximizes the evaluated cost function, using the determined first parameter value. Once the second parameter value is determined, the ultrasound system can determine a third parameter value that maximizes the evaluated cost function. In this way, the ultrasound system can sequentially (e.g., one at a time) determine the values for parameters. In another example, the ultrasound system can simultaneously determine the values for parameters by aggregating the parameters into a vector. The ultrasound system can update the vector via a stochastic gradient ascent over the vector of the evaluated cost function and can determine the parameter values through Equation (4). The gradient can be numerically determined by calculation of the change in the cost function resulting from the change in the parameter vector. Additionally, or alternatively, the ultrasound system can determine the value of parameters in the vector by searching over a finite number of choices of the parameterization, and selecting the parameters associated with the lowest evaluated cost function. Additionally, or alternatively, the system can use a binary search to determine the parameterization, by searching over ranked values of a given parameter aggregated into a vector and selecting the values based on a relative increase or decrease of the evaluated cost function.

At 1210 an additional ultrasound image with color-flow data is generated. The additional ultrasound image is generated based on the determined values of the parameters. For example, the ultrasound system 100 can generate and display another ultrasound image (e.g., after-adaptation image 904) different from the ultrasound image 120.

### Example Devices

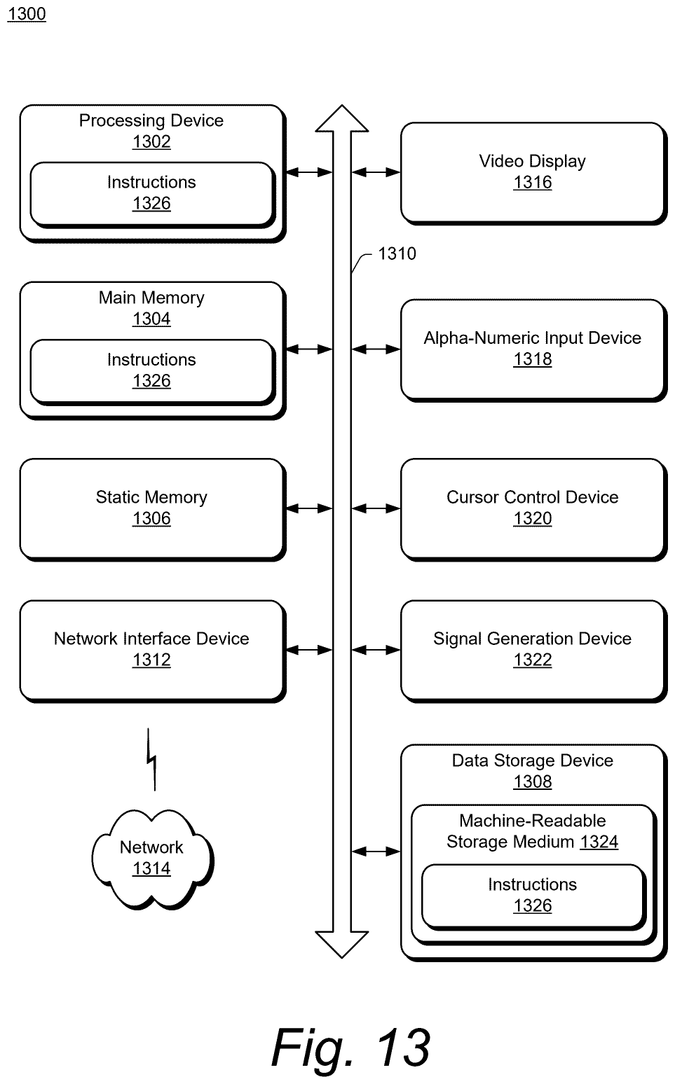

FIG. 13 illustrates a block diagram of an example computing device 1300 that can perform one or more of the operations described herein, in accordance with some implementations. The computing device 1300 can be connected to other computing devices in a local area network (LAN), an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some implementations, the computing device 1300 is one or more of an ultrasound machine, an access point, and a packet-forwarding component.

The example computing device 1300 can include a processing device 1302 (a general-purpose processor, a programmable logic device (PLD), etc.), a main memory 1304 (e.g., synchronous dynamic random-access memory (DRAM), read-only memory (ROM)), and a static memory 1306 (e.g., flash memory and a data storage device 1308), which can communicate with each other via a bus 1310. The processing device 1302 can be provided by one or more general-purpose processing devices such as a microprocessor, a central processing unit, or the like. In an illustrative example, the processing device 1302 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1302 can also comprise one or more special-purpose processing devices such as an application-specific integrated circuit (ASIC), a field programmable gate array (FPGA), a digital signal processor (DSP), a network processor, or the like. The processing device 1302 can be configured to execute the operations described herein, in accordance with one or more aspects of the present disclosure, for performing the operations and steps discussed herein.

The computing device 1300 can further include a network interface device 1312, which can communicate with a network 1314. The computing device 1300 also can include a video display unit 1316 (e.g., a liquid crystal display (LCD), organic light-emitting diode (OLED), or a cathode ray tube (CRT)), an alphanumeric input device 1318 (e.g., a keyboard), a cursor control device 1320 (e.g., a mouse), and an acoustic signal generation device 1322 (e.g., a speaker and/or a microphone). In one embodiment, the video display unit 1316, the alphanumeric input device 1318, and the cursor control device 1320 can be combined into a single component or device (e.g., an LCD touch screen).

The data storage device 1308 can include a computer-readable storage medium 1324 on which can be stored one or more sets of instructions 1326 (e.g., instructions for carrying out the operations described herein, in accordance with one or more aspects of the present disclosure). The instructions 1326 can also reside, completely or at least partially, within the main memory 1304 and/or within the processing device 1302 during execution thereof by the computing device 1300, where the main memory 1304 and the processing device 1302 also constitute computer-readable media. The instructions can further be transmitted or received over the network 1314 via the network interface device 1312.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, modules described herein (e.g., the position controller 108, fixture generator 104, etc.) are embodied in the data storage device 1308 of the computing device 1300 as executable instructions or code. Although represented as software implementations, the described modules can be implemented as any form of a control application, software application, signal-processing and control module, hardware, or firmware installed on the computing device 1300.

While the computer-readable storage medium 1324 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Conclusion

Embodiments for optimizing color imaging in ultrasounds through adaptive processes are disclosed and are advantageous, as they enable more sensitive and clear ultrasound images. The adaptive-color imaging techniques disclosed herein provide solutions that enable ultrasound systems to generate more precise and useful ultrasound images of internal organs, which may, in turn, deliver more accurate clinical assessments and diagnoses.

What is claimed is:

1. A method comprising:
receiving a first user selection that selects one or more processes from a process group;
receiving a second user selection that selects feedback data from a data group;
adapting parameters of the one or more processes based on a cost function of the feedback data; and
generating an ultrasound image, the ultrasound image having color-flow data based on the parameters.

2. The method of claim 1, wherein the process group includes a transmitting process, a receiving process, and a color-imaging process.

3. The method of claim 1, wherein the data group includes radiofrequency data and color-image data.

4. The method of claim 1, further comprising, in response to receiving the first user selection:
displaying a process group panel, the process group panel having parameters associated with the one or more processes.

5. The method of claim 1, further comprising, in response to receiving the second user selection:
displaying a data group panel, the data group panel having properties associated with the feedback data.

6. The method of claim 1, further comprising, in response to generating the ultrasound image:
generating similarity scores, the similarity scores generated based on comparing the ultrasound image to a plurality of additional ultrasound images;
selecting a target ultrasound image from the plurality of additional ultrasound images, the target ultrasound image selected based on the similarity scores;
obtaining parameter values of the one or more processes from the process group; and
initializing an adaptation process with the parameter values.

7. The method of claim 1, further comprising:
adapting the parameter values through the adaptation process by using parameter values from the target ultrasound image; and
generating an updated ultrasound image, the updated ultrasound image having updated color-flow data based on the parameter values.

8. An ultrasound system comprising:
an ultrasound scanner configured to generate ultrasound images based on echoes of ultrasound signals transmitted by the ultrasound scanner into a subject at one or more anatomical targets of interest;
a display device configured to display a user interface including an ultrasound-control panel, an ultrasound-image panel, and a process-selection panel;
one or more processors; and
one or more computer-readable storage media having instructions stored thereon that, responsive to execution by the one or more processors, cause the one or more processors to:
determine weights for regions of a color box associated with an ultrasound image;
determine a cost function associated with the ultrasound image;
evaluate the cost function associated with the ultrasound image;
determine values for parameters based on the evaluation; and
generate, based on the values of the parameters, an additional ultrasound image with color-flow data.

9. The ultrasound system of claim 8, wherein the ultrasound-control panel includes selectable controls for gain, depth, image-saving, examination presets, and configuration-storage.

10. The ultrasound system of claim 8, wherein the ultrasound-image panel displays ultrasound images.

11. The ultrasound system of claim 8, wherein the process-selection panel includes selectable controls for the process group and the data group, and the selectable controls are configured to cause, responsive to selection of the selectable controls, the display device to display the process group panel and the data group panel.

12. The ultrasound system of claim 8, wherein the weights are determined based on a cost function of the weights.

13. The ultrasound system of claim 8, wherein the cost function associated with the ultrasound image is determined based on ultrasound data associated with the ultrasound image or on color-image data generated from the ultrasound data.

14. The ultrasound system of claim 8, wherein the cost function associated with the ultrasound image is evaluated over multiple values of parameters associated with the process group.

15. The ultrasound system of claim 8, wherein the parameters are used to generate the ultrasound data associated with the ultrasound image.

16. The ultrasound system of claim 8, wherein the values for the parameters are determined sequentially.

17. The ultrasound system of claim 8, wherein the values for the parameters are determined simultaneously, the values are based on a vector, and the vector includes two or more parameter values.

18. The ultrasound system of claim 8, wherein the one or more processors are further configured to:
generate similarity scores based on a comparison between the ultrasound image and a plurality of additional ultrasound images;
select a target ultrasound image from the plurality of additional ultrasound images, the target ultrasound image selected based on the similarity scores;
obtain parameter values of the one or more processes from the process group; and
initialize an adaptation process with the parameter values.

19. The ultrasound system of claim 8, wherein the one or more processors are further configured to:
adapt the parameter values through the adaptation process by using parameter values from the target ultrasound image; and
generate an updated ultrasound image, the updated ultrasound image having updated color-flow data based on the parameter values.

20. A method comprising:
generating an ultrasound image, the ultrasound image having color-flow data;
generating similarity scores, the similarity scores generated based on comparing the ultrasound image to a plurality of additional ultrasound images;
selecting a target ultrasound image from the plurality of additional ultrasound images, the target ultrasound image selected based on the similarity scores;
obtaining parameter values of the one or more processes from the process group;
initializing an adaptation process with the parameter values;
adapting the parameter values through the adaptation process by using parameter values from the target ultrasound image; and
generating an updated ultrasound image, the updated ultrasound image having updated color-flow data based on the parameter values.
